(19) United States
(12) Patent Application Publication
(10) Pub. No.: US 2023/0116505 A1
(43) Pub. Date: Apr. 13, 2023

(54) ELECTROCHEMICAL SENSOR AND METHOD FOR MANUFACTURING SAME

(71) Applicant: DONGWOO FINE-CHEM CO., LTD., Jeollabuk-do (KR)

(72) Inventors: Seung-Hwan CHON, Yongin (KR); Dong-Ok KIM, Pyeongtaek (KR); Young-Keun LEE, Goyang (KR); Soo-Ho CHO, Hwaseong (KR)

(21) Appl. No.: 17/909,404

(22) PCT Filed: Mar. 2, 2021
(86) PCT No.: PCT/KR2021/002575

§ 371 Date: Sep. 5, 2022

(30) Foreign Application Priority Data
Mar. 4, 2020 (KR) 10-2020-0027319

(51) Int. Cl. G01N 27/27; G01N 27/416; G01N 27/327

(52) U.S. Cl. CPC G01N 27/27; G01N 27/3275; G01N 27/4163

(57) ABSTRACT

The present disclosure relates to an electrochemical sensor including: a substrate: a plurality of working electrodes formed on the substrate; and a single reference electrode formed on the substrate, wherein a separation distance between the single reference electrode and the plurality of working electrodes formed around the reference electrode satisfies Equation 1 below, and a method for manufacturing the same.

$$\text{50} μ \text{} \text{m} \leq \text{Distance between the electrodes} \leq \text{5} \text{mm}$$

### TECHNICAL FIELD

The present disclosure relates to an electrochemical sensor and a method for manufacturing the same.

### BACKGROUND ART

A technology for detecting pathogens in the human body with high sensitivity and specificity is a very necessary technology for early diagnosis and disease treatment. The disease is diagnosed at an early stage, thereby significantly reducing the cost burden that may occur due to worsening of the patients‘ diseases and further increasing the effectiveness of treatment.

The sensor may be divided into a two-electrode system consisting of a working electrode whose potential is changed according to the concentration of components in the measurement solution, thereby having a purpose of allowing a current to flow during an electrode reaction and a reference electrode that has a constant potential and is a reference of the potential for obtaining the generated potential of the working electrode, a three-electrode system further including a counter electrode that sends or receives a current so that the reaction occurs on the surface of the working electrode, and the like.

A typical sensor has only one sensor within the strip. For this reason, there is a problem in that there is a possibility of receiving a wrong measurement signal due to a user’s sampling error and manufacturing defects.

In order to solve such a problem, conventional sensors have been researched on a method of introducing a plurality of electrode systems, but the electrode systems applied to the conventional sensors have had a problem in that one or more reference electrodes are required to detect one or more components contained in a sample. For example, the conventional art has had problems in that it has been necessary to provide with a reference electrode for measurement in each detection unit in order to detect various types of antigens, and when multiple reference electrodes are used, it is difficult to store the electrode systems or a biosensor including the same, and measurement deviation increases.

In order to solve such problems, Korean Pat. No. 10-1541798 discloses a technique for correcting measurement errors according to changes in conditions through measurement of a reference solution with a known oxidation-reduction potential, but there is a problem in that it is difficult to use the technique in a general-purpose electrochemical sensor in that it includes a site capable of specific binding to glycated hemoglobin and contains molecules whose oxidation-reduction reaction potential is changed by glycated hemoglobin binding without external supply of voltage and current.

### DISCLOSURE

### Technical Problem

In order to solve such problems, an object of the present disclosure is to provide an electrochemical sensor capable of improving measurement accuracy and measurement precision by forming a plurality of working electrodes using the same reference electrode at a predetermined separation distance around the reference electrode, thereby obtaining and analyzing a plurality of measurement signals to minimize the measurement dispersion, and a method for manufacturing the same.

### Technical Solution

The present disclosure provides an electrochemical sensor including: a substrate; a plurality of working electrodes formed on the substrate; and a single reference electrode formed on the substrate, wherein a distance between one or more of the plurality of working electrodes and the reference electrode satisfies Equation 1 below.

Furthermore, the present disclosure provides a method for manufacturing an electrochemical sensor, the method comprising steps of: (a) forming a plurality of working electrodes on a substrate; and (b) forming a single reference electrode on the substrate, wherein a distance between one or more of the plurality of working electrodes and the reference electrode satisfies Equation 1 below.

$$50 \text{μ} \text{} \text{m} \leq \text{Distance between the electrodes} \leq \text{5mm}$$

### Advantageous Effects

The electrochemical sensor according to the present disclosure enables the realization of an electrochemical sensor for improving measurement accuracy and measurement precision by forming a plurality of working electrodes, thereby obtaining and analyzing a plurality of measurement signals at a time to minimize measurement dispersion.

Further, the electrochemical sensor according to the present disclosure has a further improved effect in that the plurality of working electrodes can measure different substances respectively, the measurement time is shortened, and the measurement procedure is simplified.

Moreover, since the plurality of working electrodes surrounding the reference electrode have a predetermined separation distance from the reference electrode, the electrochemical sensor according to the present disclosure has an advantage in that measurement dispersion can be minimized.

### DESCRIPTION OF DRAWINGS

FIG. 1 is a plan view of an electrochemical sensor according to one embodiment of the present disclosure.

FIG. 2 is a view showing a working electrode arrangement form according to one embodiment of the present disclosure.

FIG. 3 is graphs showing the measurement results of Example 1 and Comparative Example 1.

### BEST MODE FOR CARRYING OUT THE INVENTION

The present disclosure is an invention relating to an electrochemical sensor including a plurality of working electrodes having a predetermined separation distance around a single reference electrode, and includes a method for manufacturing the electrochemical sensor. The electrochemical sensor according to the present disclosure includes a plurality of working electrodes having a predetermined separation distance around a single reference electrode, thereby enabling measurement errors to be minimized by a method of obtaining an average or median value excluding the maximum and minimum values by obtaining a plurality of measurement signals at once. Further, the electrochemical sensor according to the present disclosure has an advantage in that the measurement time can be shortened and the measurement procedure can be simplified by enabling two or more different substances to be analyzed at the same time.

In the electrochemical sensor according to the present disclosure and the method for manufacturing the same, the detection target sample may be a biological sample such as blood, body fluid, urine, saliva, tears, sweat, etc., and may be other liquid samples, but the present disclosure is not limited thereto.

In the electrochemical sensor according to the present disclosure and the method for manufacturing the same, a detection target substance may be glucose, lactate, cholesterol, vitamin C (ascorbic acid), alcohol, various cations, or various anions, but the present disclosure is not limited thereto.

More specifically, the present disclosure relates to an electrochemical sensor including: a substrate; a plurality of working electrodes formed on the substrate; and a single reference electrode formed on the substrate, wherein a distance between one or more of the plurality of working electrodes and the reference electrode satisfies Equation 1 below.

Further, the present disclosure relates to a method for manufacturing an electrochemical sensor, the method comprising steps of: (a) forming a plurality of working electrodes on a substrate; and (b) forming a single reference electrode on the substrate, wherein a distance between one or more of the plurality of working electrodes and the reference electrode satisfies Equation 1 below.

$$50 \text{μ} \text{} \text{m} \leq \text{Distance between the electrodes} \leq \text{5mm}$$

Hereinafter, the advantages and features of the present disclosure, and a method for achieving them will become clear with reference to the embodiments described below in detail in conjunction with the accompanying drawings.

### Electrochemical Sensor

The electrochemical sensor according to the present disclosure is not particularly limited as long as it is an electrochemical sensor including working electrodes and a reference electrode, and particularly, the electrochemical sensor according to the present disclosure is characterized in that it includes a plurality of working electrodes having a predetermined separation distance around a single reference electrode.

Specifically, FIG. 1 is a plan view schematically illustrating an electrochemical sensor including one reference electrode 30 and four working electrodes 20 according to one embodiment of the present disclosure. Referring to FIG. 1, the electrochemical sensor according to the present disclosure may include working electrodes 20, a reference electrode 30, and a wiring part 40 which are provided on the upper surface of a substrate 10, and the working electrodes 20 may have an enzyme reaction layer (not shown) provided on the upper surface thereof. The four working electrodes 20 may include working electrodes 20 which have a square-shaped figure formed by an imaginary line connecting each working electrode 20, and which are formed around one reference electrode 30 at a separation distance of 1 mm.

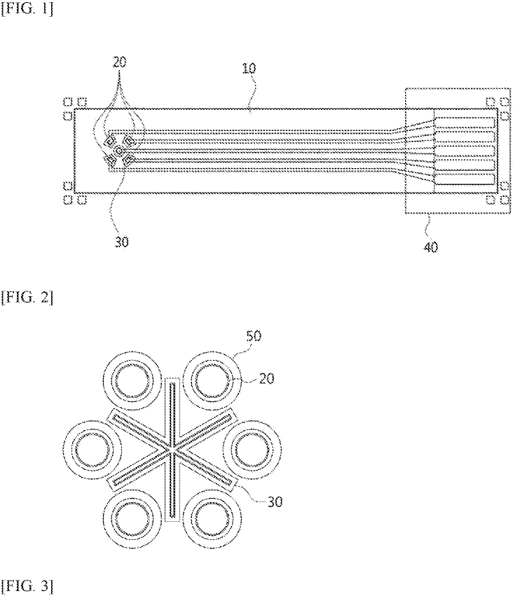

FIG. 2 is a view showing an electrode arrangement form including six working electrodes 20 according to one embodiment of the present disclosure. Referring to FIG. 2, the six working electrodes 20 included in the electrochemical sensor according to the present disclosure may include working electrodes 20 which have a regular hexagonal figure formed by an imaginary line connecting each working electrode 20, and which are formed around the reference electrode 30 at a separation distance of 0.5 mm. An enzyme reaction layer 50 may be provided on the upper surface of the working electrodes 20.

The plurality of working electrodes included in the electrochemical sensor according to the present disclosure are not limited to a constant arrangement form, and may be arranged in various shapes such as linear and polygonal shapes as well as the shapes shown in FIGS. 1 and 2

Hereinafter, the structure of the electrochemical sensor according to the present disclosure will be described for each configuration.

### Substrate

The substrate serves to provide a structural base of the components constituting the electrochemical sensor.

Examples of a material for the substrate according to the present disclosure may include conventional materials or materials developed later, but the present disclosure is not particularly limited thereto. Examples of the material of the substrate according to the present disclosure may include films composed of silicon, glass, glass epoxy, ceramics, and thermoplastic resins including: polyester-based resins such as polyethylene terephthalate (PET), and polybutylene terephthalate, cellulose-based resins such as diacetyl cellulose and triacetyl cellulose: polycarbonate-based resins: acrylic resins such as polymethyl methacrylate and polyethyl methacrylate; styrene-based resins such as polystyrene and an acrylonitrile-styrene copolymer; polyolefin-based resins such as polyethylene, polypropylene, polyolefin having a cyclo-based or norbornene structure, and an ethylene-propylene copolymer; vinyl chloride-based resins; amide-based resins such as nylon and aromatic polyamide; imide-based resins; polyether sulfone-based resins; sulfone-based resins; polyether ether ketone-based resins; polyphenylene sulfide-based resins; vinyl alcohol-based resins; vinylidene chloride-based resins; polyvinyl butyral resins; allylate-based resins; polyoxymethylene-based resins; and epoxy-based resins, and may also include films composed of blends of the above-mentioned thermoplastic resins. Further, examples of the material for the substrate according to the present disclosure may further include one or more of polyimide and films formed of thermosetting resins or UV curable resins such as methacrylic resins, urethane-based resins, acrylic urethane-based resins, epoxy-based resins, and silicone-based resins, but the present disclosure is not limited thereto.

### Reference Electrode

The reference electrode is an electrode that has a constant potential and is a reference of the potential for obtaining the generated potential of the working electrodes, and may include one or more selected from the group consisting of a silver-silver chloride (Ag/AgCI) electrode, a calomel electrode, a mercury-mercury sulfate electrode, and a mercury-mercury oxide electrode, etc. It is preferable that the reference electrode is a silver-silver chloride (Ag/AgCl) electrode considering that it has less hysteresis of the potential with respect to the temperature cycle, and the potential is stable up to high temperatures. Further, it may also possible to include the same carbon paste used for the working electrodes below.

The single reference electrode included in the electrochemical sensor according to the present disclosure may mean one reference electrode.

### Working Electrodes

The working electrode is an electrode in which a reaction occurs, has the purpose of allowing a current to flow during an electrode reaction, and may also be also called as an anode/cathode depending on whether the reaction occurring in the electrode is an oxidation/reduction reaction. In one or more embodiments, the working electrode may include one or more selected from the group consisting of: gold (Au); silver (Ag); copper (Cu); platinum (Pt): titanium (Ti); nickel (Ni); tin (Sn); molybdenum (Mo); palladium (Pd); cobalt (Co); alloys thereof); pyrolytic graphite; glassy carbon; carbon paste; perfluorocarbon (PFC); and carbon nanotube (CNT), but it is preferably carbon paste in consideration of ease of manufacture, excellent reproducibility, and a wide potential window in the oxidation/reduction direction. The materials used for the working electrode may be used alone, or two or more materials may be used as a multilayer film. As an example, a material used as the conducting wire of the working electrode may be preferably a silver-palladium-copper alloy (Ag-Pd-Cu; APC), the working electrode may be a carbon paste, and the electrode protective layer is preferably Indium Tin Oxide (ITO) or Indium Zinc Oxide (IZO).

In the electrochemical sensor according to the present disclosure, the plurality of working electrodes may mean two, three, four, or five or more working electrodes.

One or more of the plurality of working electrodes may be formed around the single reference electrode at a predetermined separation distance, and “distance between the electrodes”, which means a distance from one or more of the plurality of working electrodes to the reference electrode, satisfies [Equation 1] below.

$$50 \text{μ} \text{} \text{m} \leq \text{Distance between the electrodes} \leq \text{5mm}$$

As shown in Equation 1, the distance between the electrodes of the electrochemical sensor according to the present disclosure may be 50 µm to 5 mm, preferably 200 µm to 3.0 mm, and more preferably 300 µm to 2.0 mm. If the distance between the working electrode and the reference electrode is less than 50 µm, there is a possibility that measurement errors may occur due to a short circuit between the reference electrode and the working electrodes, and if it exceeds 5 mm, the measured value becomes smaller due to a delay in the measurement time of the sensor and an increase in solution resistance.

### Counter Electrode

The electrochemical sensor according to the present disclosure may further include a counter electrode or an electrode protective layer in addition to the working electrodes and the reference electrode

The counter electrode serves to send or receive a current so that a reaction occurs on the surface of the working electrodes. That is, while the flow of current is mainly exchanged between the counter electrode and the working electrodes, oxidation and reduction reactions occur. At this time, the reference electrode serves as a feedback sensor to measure and monitor the potential of the counter electrode and maintain it in a constant state. For the counter electrode, all materials described in the above item working electrodes and the above item reference electrode may be used, and it is preferable to use the same material as the working electrodes and/or the reference electrode for process simplification and manufacturing cost improvement.

### Method for Manufacturing Electrochemical Sensor

The method for manufacturing an electrochemical sensor according to the present disclosure may comprise steps of: (a) forming a plurality of working electrodes on a substrate; and/or (b) forming a single reference electrode on the substrate, wherein a distance between one or more of the plurality of working electrodes and the reference electrode may satisfy Equation 1 below.

$$50 \text{μ} \text{} \text{m} \leq \text{Distance between the electrodes} \leq \text{5mm}$$

The steps (a) and (b) may be performed by including one or more processes selected from the group consisting of screen printing, letterpress printing, engraving printing, lithographic printing, and photolithography. In one or more embodiments, it is preferable to form a wiring part by performing a photolithography process in the steps (a) and (b), and each electrode may be formed by any one selected from the group consisting of screen printing, letterpress printing, engraving printing, and lithographic printing, and may be preferably formed by screen printing.

The photolithography may be a method capable of integrally forming wiring by forming a carbon paste and/or a metal film on a substrate and patterning this through a mask.

The electrochemical sensor manufactured by the manufacturing method may exhibit all the characteristics described in the item <Electrochemical sensor>.

### Method of Measuring Electrochemical Signal

The present disclosure includes a method for measuring an electrochemical signal of a detection target substance using the electrochemical sensor and/or an electrochemical sensor manufactured by the method for manufacturing the electrochemical sensor. The method of measuring an electrochemical signal according to the present disclosure can measure electrochemical signals of a plurality of detection target substances in a short time by simultaneously analyzing two or more different substances. Further, it is possible to minimize measurement errors by a method of obtaining a plurality of measurement signals obtained from the plurality of working electrodes and then obtaining an average or median value excluding the maximum and minimum values.

In the present specification, electrochemically measure refers to measurement by applying an electrochemical measurement method, and in one or more embodiments, the electrochemical measurement method may include an amperometric method, a potentiometric measurement method, a coulometric analysis method, etc., and may be preferably an amperometric method.

The electrochemical sensor according to the present disclosure and/or the electrochemical sensor manufactured by the method for manufacturing the electrochemical sensor may be ones analyzing two or more different substances at the same time.

Further, the electrochemical sensor may be one capable of minimizing measurement errors by a method of obtaining an average or median value excluding the maximum and minimum values after obtaining a plurality of measurement signals.

In another embodiment, the method for measuring an electrochemical signal of a detection target substance according to the present disclosure may comprise bringing the detection target substance into contact with the reagent and then applying a voltage to an electrode unit including the working electrodes and the reference electrode, measuring a response current value emitted during the application, and calculating an electrochemical signal of the detection target substance in the sample based on the response current value. In one or more embodiments, the applied voltage is not particularly limited, but it may be -500 to +500 mV, preferably -200 to +200 mV. based on the silver-silver chloride electrode (Ag/AgCl electrode).

In another embodiment, the method for measuring an electrochemical signal of a detection target substance according to the present disclosure may comprise bringing the detection target substance into contact with the reagent and maintaining it in the non-application state for a predetermined period of time, and then applying a voltage to the electrode unit, or bringing the detection target substance into contact with the reagent and applying a voltage to the electrode unit at the same time.

The method for measuring an electrochemical signal of a detection target substance according to the present disclosure may measure response current values discharged respectively by bringing a sample including the detection target substance into contact with portions of some of the working electrodes, bringing another sample including a detection target substance different from the detection target substance into contact with portions of the remaining working electrodes, and then applying a voltage to the electrode unit.

The method for measuring an electrochemical signal of a detection target substance according to the present disclosure may be one in which a voltage is applied to the electrode unit after bringing a sample into contact with the plurality of working electrodes, one in which a voltage is applied to the electrode unit, one in which a plurality of response current values discharged during the application are measured, one in which an average value excluding the maximum and minimum values is measured after acquiring the plurality of response current values, or one in which measurement errors can be minimized by a method of obtaining a median value excluding the maximum and minimum values after acquiring the plurality of response current values.

### Electrochemical Signal Measurement System

In another embodiment, the present disclosure relates to an electrochemical signal measurement system for measuring an electrochemical signal of a detection target substance in a sample, the electrochemical signal measurement system including the electrochemical sensor according to the present disclosure, a means for applying a voltage to an electrode unit of the electrochemical sensor, and a means for measuring a current in the electrode unit. According to a system for measuring an electrochemical signal of a detection target substance according to the present disclosure can measure multiple substances in a short time by simultaneously analyzing two or more different substances in order to measure the electrochemical signal of the detection target substance in the sample using the electrochemical sensor according to the present disclosure. Further, it can minimize the measurement errors and shorten the measurement time by using a method of obtaining an average or median value excluding the maximum and minimum values after acquiring a plurality of measurement signals at the same time.

The application means is not particularly limited as long as it conducts with the electrode unit of the electrochemical sensor and can apply a voltage, and a known application means may be used. In one or more embodiments, the application means may include a contact capable of coming into contact with the electrode unit of the electrochemical sensor, and a power source such as a DC power supply.

The measuring means is for measuring a plurality of currents in the electrode unit generated during voltage application, and in one or more embodiments, it may include one capable of measuring a response current value correlating with the amount of electrons emitted from the electrode unit of the electrochemical sensor, and one which is used as a conventional or later developed electrochemical sensor may be used

### Mode for Carrying Out the Invention

Hereinafter, embodiments of the present disclosure will be specifically described. However, the present disclosure is not limited to the embodiments disclosed below, but may be implemented in various different forms, and only the present embodiments are provided to allow the disclosure of the present disclosure to be complete, and to completely inform those with ordinary skill in the art to which the present disclosure pertains of the scope of the invention, and the present disclosure is only defined by the scope of the claims. The same reference numerals refer to the same elements throughout the specification.

The terms used in the present specification is for describing the embodiments, and is not intended to limit the present disclosure. In the present specification, the singular forms also include the plural forms unless specifically stated otherwise in the phrases.

The term, comprises and/or comprising used in the specification, is used in a sense that it does not exclude the existence or addition of one or more other components, steps, operations and/or elements other than the mentioned component, step, operation and/or element.

Hereinafter, preferred embodiments of the present disclosure will be described in detail as follows.

### Examples and Comparative Examples

Electrochemical sensors corresponding to Examples and Comparative Examples were manufactured through the manufacturing method below.

### Example 1

As shown below, a glucose sensor of Example 1 of the same structure as the glucose sensor shown in FIG. 1 was manufactured.

After an Ag alloy layer with a thickness of about 2,000 A and an IZO metal protective layer with a thickness of about 500 Å were patterned on a substrate using a photolithography method, a carbon paste electrode layer (containing Prussian blue) with a thickness of about 10 µm was printed by a screen printing method. Working electrodes were formed by sequentially stacking an enzyme reaction layer to which glucose oxidase was immobilized with chitosan thereon.

A biosensor was prepared by forming an Ag/AgCl reference electrode spaced apart from the working electrodes on the substrate.

In this case, a distance between the two electrodes was set to 1 mm, and a sensor having four working electrodes was fabricated as shown in FIG. 1.

### Example 2

The manufacturing method was the same as in Example 1, and a sensor having a distance between the electrodes of 0.5 mm and having four working electrodes was manufactured.

### Example 3

The manufacturing method was the same as in Example 1, and a sensor having a distance between the electrodes of 0.5 mm and having six working electrodes was manufactured.

### Comparative Example 1

The manufacturing method was the same as in Example 1, and a sensor having a distance between the electrodes of 1 mm and having one working electrode was manufactured.

### Comparative Example 2

The manufacturing method was the same as in Example 1, and a sensor having a distance between the electrodes of 10 mm and having four working electrodes was manufactured.

### Experimental Example

The sensors manufactured by the above-described Examples and Comparison Examples were measured by the following method.

As a sample, a sample in which 0.1 to 0.3 mM of glucose was dissolved in phosphate-buffered saline (PBS) was made and measured. At this time, the volume of the sample used is 30 µL, but it is not limited if it is an amount of being applied onto the reference electrode and the working electrodes as a sufficient amount applied onto the surface of the electrodes. Current values at 30 seconds obtained by measuring this using a potentiostat were converted into concentration values and shown in Table 1 below. However, they are not limited to the current values at 30 seconds, and the voltage applied at this time was -100 mV.

TABLE 1

| | Distance between the electrodes | Number of working electrodes | R² (Accuracy) | %RSD (0.2 mM Glucose) (Precision) |
|---|---|---|---|---|
| Example 1 | 1 mm | 4 | 0.991 | 4.25% |
| Example 2 | 0.5 mm | 4 | 0.995 | 3.60% |
| Example 3 | 0.5 mm | 6 | 0.996 | 2.57% |
| Comparative Example 1 | 1 mm | 1 | 0.909 | 11.9% |
| Comparative Example 2 | 10 mm | 4 | 0.875 | 15.3% |

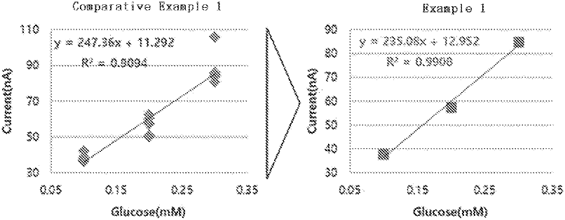

When glucose concentration values are measured using the electrochemical sensors of Examples 1 to 3 in which the distances between the electrodes correspond to the disclosed range of the present disclosure, since the coefficients of determination (R²) are 0.991 or more, it can be confirmed that the accuracy is improved compared to Comparative Example 1 with one working electrode and Comparative Example 2 in which the distance between the electrodes is outside the disclosed range of the present disclosure. Further, when looking at the percentage of the relative standard deviation (RSD), it can be confirmed that Examples 1 to 3 show excellent precision compared to Comparative Examples 1 and 2. Specifically, FIG. 3 is graphs showing the measurement results of Example 1 and Comparative Example 1, and it can be confirmed that Example 1 has improved accuracy and precision than Comparative Example 1. Therefore, it can be confirmed that the electrochemical sensor having a plurality of working electrodes and a distance between the electrodes disclosed in the present disclosure exhibits an effect of improving accuracy and precision.

### Industrial Applicability

According to the electrochemical sensor according to the present disclosure, there is an industrial applicability by forming a plurality of working electrodes with a predetermined separation distance from the reference electrode to acquire a plurality of measurement signals at a time, and enabling measurement accuracy and measurement precision to be improved by analyzing the acquired measurement signals.

1. An electrochemical sensor including:
a substrate;
a plurality of working electrodes formed on the substrate; and
a single reference electrode formed on the substrate,
wherein a distance between one or more of the plurality of working electrodes and the reference electrode satisfies Equation 1 below
$$50 μ \text{} \text{m} \leq \text{Distance} \text{between} \text{the} \text{electrodes} \leq \text{5} \text{mm}$$
.

2. The electrochemical sensor of claim 1, wherein the working electrode includes one or more selected from the group consisting of: gold (Au); silver (Ag); copper (Cu); platinum (Pt); titanium (Ti); nickel (Ni); tin (Sn); molybdenum (Mo); palladium (Pd); cobalt (Co); alloys thereof; pyrolytic graphite; glassy carbon; carbon paste; perfluorocarbon (PFC); and carbon nanotube (CNT).

3. The electrochemical sensor of claim 1, wherein the reference electrode includes one or more selected from the group consisting of a silver-silver chloride (Ag/AgCl) electrode, a calomel electrode, a mercury-mercury sulfate electrode, and a mercury-mercury oxide electrode.

4. The electrochemical sensor of claim 1, wherein the working electrodes analyze two or more different substances at the same time.

5. The electrochemical sensor of claim 1, wherein the electrochemical sensor can minimize measurement errors by a method of obtaining an average or median value excluding the maximum and minimum values after acquiring a plurality of measurement signals.

6. A method for manufacturing an electrochemical sensor, the method comprising steps of:
(a) forming a plurality of working electrodes on a substrate: and
(b) forming a single reference electrode on the substrate,
wherein a distance between one or more of the plurality of working electrodes and the reference electrode satisfies Equation 1 below
$$50 μ \text{} \text{m} \leq \text{Distance} \text{between} \text{the} \text{electrodes} \leq \text{5} \text{mm}$$
.

7. The method of claim 6, wherein the steps (a) and (b) include one or more processes selected from the group consisting of screen printing, letterpress printing, engraving printing, lithographic printing, and photolithography.
